(19) United States
(12) Patent Application Publication
(10) Pub. No.: US 2026/0299003 A1
(43) Pub. Date: Oct. 1, 2026

(54) MACHINE AND METHOD FOR TESTING LIGHT-EMITTING SUBSTRATE

(71) Applicant: HARVATEK CORPORATION, Hsinchu City (TW)

(72) Inventors: GUAN-YU LI, Hsinchu City (TW); YU-CHANG HU, Hsinchu City (TW); ZONG-LIANG CHEN, Hsinchu City (TW); Hsin-I Lu, HSINCHU COUNTY (TW); SHYI-MING PAN, Miaoli County (TW); FENG-HUI CHUANG, New Taipei City (TW)

(21) Appl. No.: 19/454,408

(22) Filed: Jan. 21, 2026

(30) Foreign Application Priority Data
Feb. 5, 2025 (TW) 114104062

(51) Int. Cl. G01R 31/26

(52) U.S. Cl. CPC G01R 31/2635

(57) ABSTRACT

A machine and method for testing a light-emitting substrate. In the method, a test board is provided in a test region and includes a first conductive region and a second conductive region having different electrical characteristics. Afterwards, an anisotropic conductive layer is disposed on the test board and covers the first conductive region and the second conductive region. Then, a light-emitting substrate is held in the test region, and one of the light-emitting substrate and the test board is driven to move toward the other, such that the anisotropic conductive layer is pressed between the light-emitting substrate and the test board to establish signal conduction. Finally, a test signal is provided to the light-emitting substrate through the test board.

### CROSS-REFERENCE TO RELATED PATENT APPLICATION

This application claims the benefit of priority to Taiwan Patent Application No. 114104062, filed on Feb. 5, 2025. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to light-emitting component testing, and more particularly to a machine and method for testing a light-emitting substrate.

### BACKGROUND OF THE DISCLOSURE

A light-emitting diode (LED) is a light-emitting component capable of converting electrical energy into light energy, and it has advantages such as small size, high luminous efficiency, long lifetime, high reliability, fast response time, energy saving, and environmental friendliness, and is widely applied in the fields of illumination, display, and communication. Since the performance of an LED directly affects the results in its practical application, it is important to conduct accurate testing of the LED so as to ensure its performance.

Micro light-emitting diodes (micro-LEDs) have excellent display performance and have been applied to next-generation display technology. However, micro-LEDs are small in size (less than 100 micrometers), and are large in quantity and arranged at high density per unit area, causing testing to be more difficult and less efficient. For example, in the conventional means, a probe is used to contact electrodes of chips to be tested, and voltage is applied to light up micro-LEDs for testing. However, testing every chip to be tested on an entire light-emitting substrate may take several hours or even hundreds of hours. Although chips to be tested can be powered simultaneously through an interposer, structural height differences may result in the inability to light up some of the chips.

### SUMMARY OF THE DISCLOSURE

In response to the above-referenced technical inadequacies, the present disclosure provides a machine and method for testing a light-emitting substrate, which can meet the requirements of performance testing for light-emitting diodes that are small in size, large in quantity, and arranged at high density, and can provide an economical, reliable, and efficient testing environment.

In order to solve the above-mentioned problems, one of the technical aspects adopted by the present disclosure is to provide a method for testing a light-emitting substrate, which includes: providing a test board in a test region, in which the test board includes a first conductive region and a second conductive region having different electrical characteristics; disposing an anisotropic conductive layer on the test board, in which the anisotropic conductive layer covers the first conductive region and the second conductive region; holding a light-emitting substrate at a position aligned with the test region, and driving one of the light-emitting substrate and the test board to move toward the other, such that the anisotropic conductive layer is pressed between the light-emitting substrate and the test board to establish signal conduction; and providing a test signal to the light-emitting substrate through the test board. The light-emitting substrate includes a first conductivity-type semiconductor layer and a plurality of light-emitting units and a first conductive structure formed on the first conductivity-type semiconductor layer. The first conductive structure is located on one side of the light-emitting units. Each of the light-emitting units includes a light-emitting layer disposed on the first conductivity-type semiconductor layer, a second conductivity-type semiconductor layer disposed on the light-emitting layer, and a second conductive structure disposed on the second conductivity-type semiconductor layer. The test signal includes a first test signal and a second test signal. The first test signal sequentially passes through the first conductive region of the test board and the anisotropic conductive layer to be transmitted to the first conductive structure of the light-emitting substrate. The second test signal sequentially passes through the second conductive region of the test board and the anisotropic conductive layer to be transmitted to the second conductive structure of each of the light-emitting units of the light-emitting substrate.

In one of the possible or preferred embodiments, the step of providing the test signal to the light-emitting substrate includes obtaining at least one light-emitting characteristic parameter of each of the light-emitting units of the light-emitting substrate through a light sensor.

In one of the possible or preferred embodiments, in the step of establishing the signal conduction between the light-emitting substrate and the test board through the anisotropic conductive layer, the test board remains stationary and the light-emitting substrate is driven to move toward the test board, such that the anisotropic conductive layer is pressed between the light-emitting substrate and the test board.

In one of the possible or preferred embodiments, in the step of establishing the signal conduction between the light-emitting substrate and the test board through the anisotropic conductive layer, the light-emitting substrate remains stationary and the test board is driven to move toward the light-emitting substrate, such that the anisotropic conductive layer is pressed between the light-emitting substrate and the test board.

In one of the possible or preferred embodiments, when the anisotropic conductive layer is pressed between the light-emitting substrate and the test board, a first alignment mark on the light-emitting substrate is aligned with a second alignment mark on the test board.

In order to solve the above-mentioned problems, another one of the technical aspects adopted by the present disclosure is to provide a machine for testing a light-emitting substrate, which includes a test region, a test assembly, a holder, and a driver. The test assembly is disposed in the test region, and includes a test board and an anisotropic conductive layer. The test board includes a first conductive region and a second conductive region having different electrical characteristics. The anisotropic conductive layer is disposed on the test board, and covers the first conductive region and the second conductive region. The holder is configured to hold a light-emitting substrate at a position aligned with the test region. The driver is configured to drive one of the light-emitting substrate and the test board to move toward the other, such that the anisotropic conductive layer is pressed between the light-emitting substrate and the test board to establish signal conduction. The light-emitting substrate includes a first conductivity-type semiconductor layer and a plurality of light-emitting units and a first conductive structure formed on the first conductivity-type semiconductor layer. The first conductive structure is located on one side of the light-emitting units. Each of the light-emitting units includes a light-emitting layer disposed on the first conductivity-type semiconductor layer, a second conductivity-type semiconductor layer disposed on the light-emitting layer, and a second conductive structure disposed on the second conductivity-type semiconductor layer. The test board is configured to provide a test signal to the light-emitting substrate, which includes a first test signal and a second test signal. The first test signal sequentially passes through the first conductive region of the test board and the anisotropic conductive layer to be transmitted to the first conductive structure of the light-emitting substrate. The second test signal sequentially passes through the second conductive region of the test board and the anisotropic conductive layer to be transmitted to the second conductive structure of each of the light-emitting units of the light-emitting substrate.

In one of the possible or preferred embodiments, the machine further includes a light sensor that is configured to obtain at least one light-emitting characteristic parameter of each of the light-emitting units of the light-emitting substrate.

In one of the possible or preferred embodiments, the holder is a light-permeable suction holder, and the light sensor is disposed above the light-permeable suction holder.

In one of the possible or preferred embodiments, the test board remains stationary in the test region, and the driver is drivingly connected to the holder to move the light-emitting substrate toward the test board, such that the anisotropic conductive layer is pressed between the light-emitting substrate and the test board.

In one of the possible or preferred embodiments, the light-emitting substrate remains stationary in the test region, and the driver is drivingly connected to the test assembly to move the test board toward the light-emitting substrate, such that the anisotropic conductive layer is pressed between the light-emitting substrate and the test board.

In conclusion, the machine and method for testing a light-emitting substrate can shorten testing time, simplify testing operations, reduce testing difficulty, and improve testing accuracy, by virtue of the test board including a first conductive region and a second conductive region formed in accordance with a specific layout design, the anisotropic conductive layer formed on the test board and covering the first conductive region and the second conductive region, and driving one of the light-emitting substrate and the test board to move toward the other, such that the anisotropic conductive layer is pressed between the light-emitting substrate and the test board to establish signal conduction.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view illustrating one transfer operation of a machine for testing a light-emitting substrate according to a first embodiment of the present disclosure;

FIG. 2 is a schematic view illustrating another transfer operation of the machine for testing the light-emitting substrate according to the first embodiment of the present disclosure;

FIG. 3 is a schematic view illustrating one transfer operation of a machine for testing a light-emitting substrate according to a second embodiment of the present disclosure;

FIG. 4 is a schematic view illustrating another transfer operation of the machine for testing the light-emitting substrate according to the second embodiment of the present disclosure;

FIG. 5 is a schematic side view of a light-emitting substrate to be tested;

FIG. 6 is a schematic view illustrating a test operation of the machine for testing the light-emitting substrate according to the first and second embodiments of the present disclosure, in which an anisotropic conductive layer is pressed between a light-emitting substrate and a test board to establish signal conduction;

FIG. 7 is a schematic view illustrating one transfer operation of a machine for testing a light-emitting substrate according to a third embodiment of the present disclosure;

FIG. 8 is a schematic view illustrating another transfer operation of the machine for testing the light-emitting substrate according to the third embodiment of the present disclosure; and

FIG. 9 is a flowchart of a method for testing a light-emitting substrate of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

Due to the small size, large number, and high density of light-emitting components (such as light-emitting diodes), detection becomes more difficult and less efficient in the manufacturing process of light-emitting substrates. Therefore, the present disclosure provides an innovative testing means, in which an anisotropic conductive layer is used in combination with a test board having a specially designed conductive pattern layout, the combination serving as a test assembly for testing the optoelectronic performance of the light-emitting substrates.

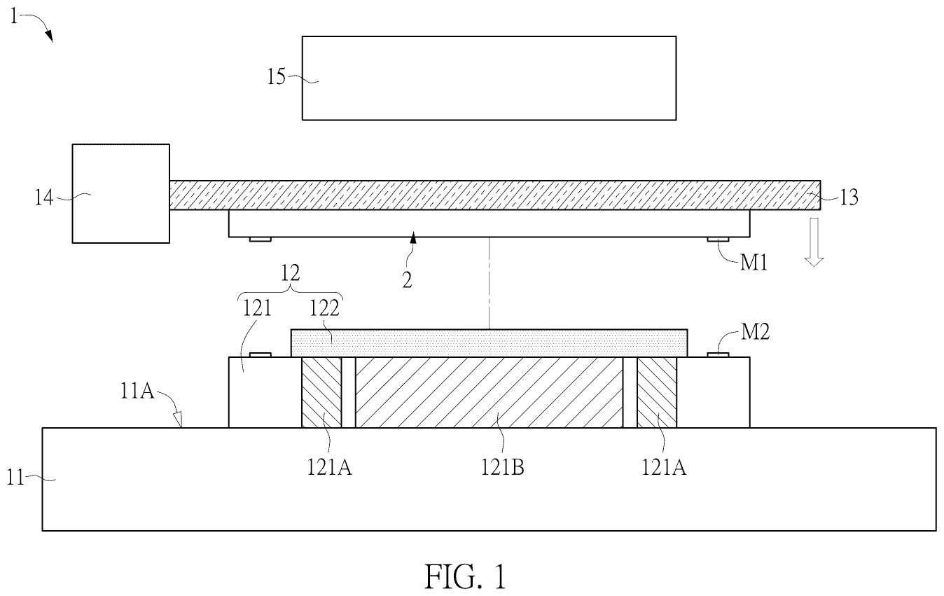

Referring to FIG. 1 to FIG. 4, the present disclosure provides a machine 1 for testing a light-emitting substrate, which includes a test region 11A, a test assembly 12, a holder 13, and a driver 14. The test assembly 12 is disposed in the test region 11A, and includes a test board 121 and an anisotropic conductive layer 122 disposed on the test board 121. The holder 13 is configured to hold a light-emitting substrate 2 in the test region 11A. The driver 14 is configured to drive one of the light-emitting substrate 2 and the test board 121 to move toward the other, such that the anisotropic conductive layer 122 is pressed between the light-emitting substrate 2 and the test board 121 to establish signal conduction. Accordingly, a test signal can be provided to the light-emitting substrate 2 through the test board 121, and the performance of the light-emitting substrate 2 can be determined according to one or more of light-emitting characteristics or electrical signals generated by the light-emitting substrate 2 in response to the test signal.

In practice, the machine 1 of the present disclosure can include a work stage that is provided with the test region 11A. Furthermore, the test assembly 12 can include a mounting fixture (not shown in the figures), by which the test board 121 and the anisotropic conductive layer 122 can be integrated into a unitary structure for use and rapidly positioned on the work stage 11. However, the above description is for exemplary purposes only, and is not meant to limit the scope of the present disclosure.

The work stage 11 can be disposed in an open space, and the test board 121 is configured to remain stationary relative to the work stage 11 or move or rotate in the test region 11A. The test board 121 includes a first conductive region 121A and a second conductive region 121B having different electrical characteristics, and the anisotropic conductive layer 2 covers the first conductive region 121A and the second conductive region 121B. In practice, the first conductive region 121A and the second conductive region 121B can have a layout design matching the light-emitting substrate 2, i.e., the patterns and positions of the first conductive region 121A and the second conductive region 121B can vary depending on the structure and arrangement of the light-emitting substrate 2. For example, the first conductive region 121A can be formed by two conductor patterns spaced apart from each other, and the second conductive region 121B can be formed by a single conductor pattern located between the two conductor patterns of the first conductive region 121A, in which conductors can be made of copper or a copper alloy. However, such examples are not intended to limit the scope of the present disclosure.

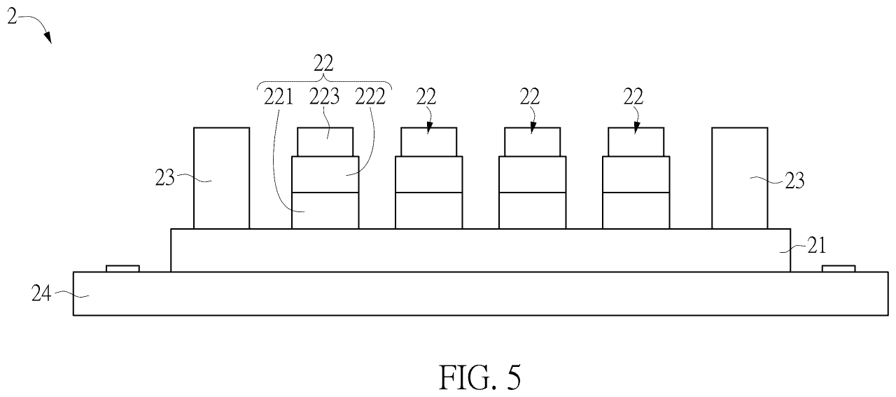

Reference is made to FIG. 5, the light-emitting substrate 2 includes a first conductivity-type semiconductor layer 21 and a plurality of light-emitting units 22 and a first conductive structure 23 formed on the first conductivity-type semiconductor layer 21. The light-emitting units 22 can be arranged in an array on the first conductivity-type semiconductor layer 21. The first conductive structure 23 can be located on one side of the light-emitting units 22. Each of the light-emitting units 22 includes a light-emitting layer 221, a second conductivity-type semiconductor layer 222, and a second conductive structure 223 that are sequentially formed on the first conductivity-type semiconductor layer 21. The first conductive structure 23 can serve as an n-type electrode, and the second conductive structure 223 can serve as a p-type electrode.

In practice, the first conductivity-type semiconductor layer 21, the light-emitting units 22, and the first conductive structure 23 can be grown on a substrate 24 (such as a sapphire substrate). The first conductivity-type semiconductor layer 21 can be a gallium nitride (GaN)-based n-type semiconductor layer, and the second conductivity-type semiconductor layer 222 of each of the light-emitting units 22 can be a gallium nitride-based p-type semiconductor layer. The light-emitting layer 221 of each of the light-emitting units 22 can be a quantum well layer. The first conductive structure 23 and the second conductive structures 223 of the light-emitting units 22 can each be a contact pad formed from a transparent conductive material (such as indium tin oxide). However, such examples are not intended to limit the scope of the present disclosure.

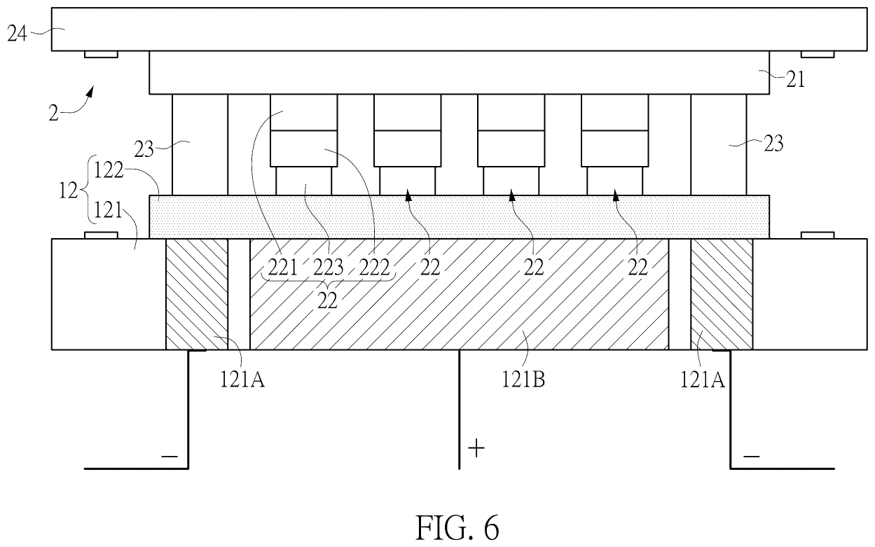

Reference is made to FIG. 6, when the anisotropic conductive layer 122 is pressed between the light-emitting substrate 2 and the test board 121, a first test signal (such as a negative voltage) can be provided from the first conductive region 121A of the test board 121, and a second test signal (such as a positive voltage) can be provided from the second conductive region 121B of the test board 121. The first test signal passes through the anisotropic conductive layer 122 to be transmitted to the first conductive structure 23 of the light-emitting substrate 2. The second test signal passes through the anisotropic conductive layer 122 to be transmitted to the second conductive structure 223 of each of the light-emitting units 22. Accordingly, each of the light-emitting units 22 can emit light for performance testing.

It is worth mentioning that the anisotropic conductive layer 122 is formed from an anisotropic conductive material, and therefore exhibits electrical conduction in the Z direction and insulation in the X and Y directions. The anisotropic conductive layer 122 is elastic such that, when compressed, it can be in close contact with the first conductive structure 23 and the second conductive structures 223, and can extend to cover a side portion of the first conductive structure 23 or the second conductive structure 223. Therefore, during testing, the anisotropic conductive layer 122 can maintain stable and reliable contact with the first and second conductive structures 23, 223 and provide good electrical conduction without being affected by differences in structural height.

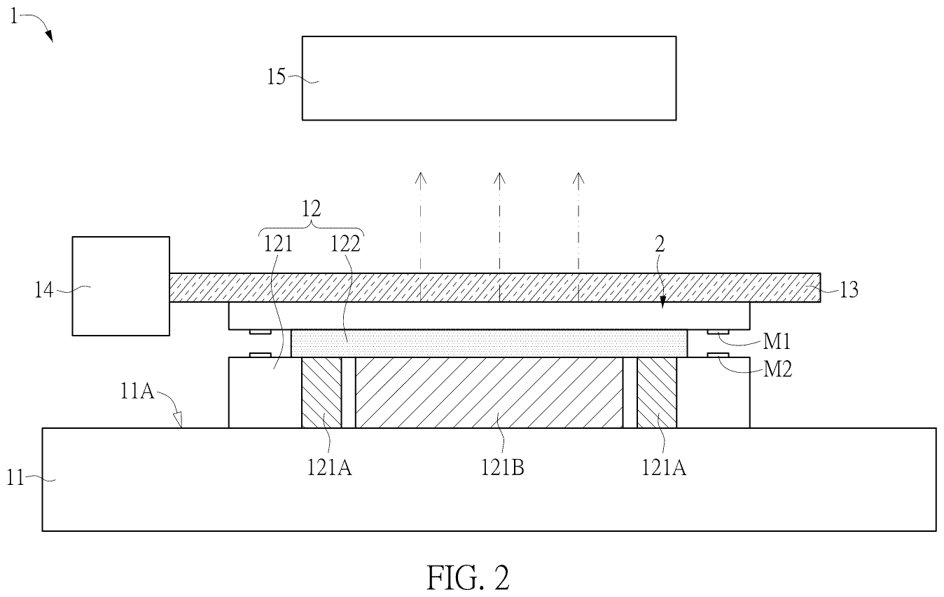

In the test operation of one possible embodiment of the present disclosure, as shown in FIG. 1 and FIG. 2, the test board 121 remains stationary in the test region 11A, and the driver 14 is drivingly connected to the holder 13. Accordingly, the holder 13 can be driven to move the light-emitting substrate 2 toward the test board 121 (i.e., downward), such that the anisotropic conductive layer 122 is pressed between the light-emitting substrate 2 and the test board 121.

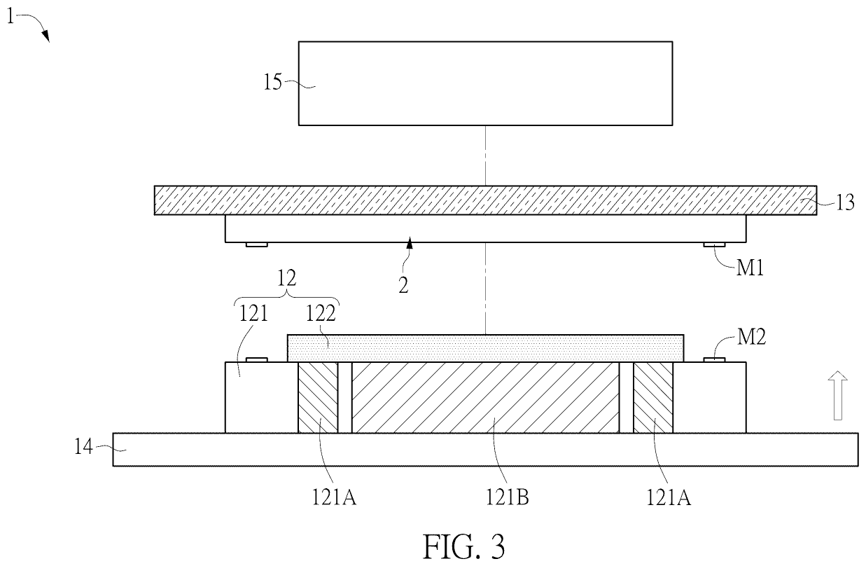

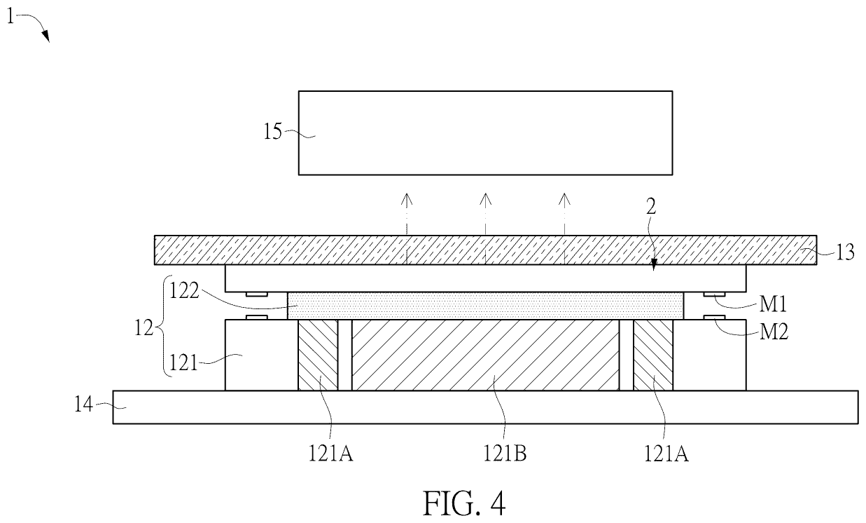

In the test operation of another possible embodiment of the present disclosure, as shown in FIG. 3 and FIG. 4, the light-emitting substrate 2 remains stationary in the test region 11A, and the driver 14 is drivingly connected to the test assembly 12, for example, to a mounting fixture of the test assembly 12 (not shown in FIGS. 3 and 4). Accordingly, the test board 121 can be driven to move toward the light-emitting substrate 2 (i.e., upward), such that the anisotropic conductive layer 122 is pressed between the light-emitting substrate 2 and the test board 121.

In practice, a first alignment mark M1 can be formed on the light-emitting substrate 2, and a second alignment mark M2 can be formed on the test board 121, in which the first alignment mark M1 corresponds in position to the second alignment mark M2. Accordingly, in the testing operation, precise vertical alignment can be achieved by aligning the first alignment mark M1 on the light-emitting substrate 2 with the second alignment mark M2 on the test board 121.

In the present disclosure, the machine 1 can further include a light sensor 15 that is configured to obtain at least one light-emitting characteristic parameter of each of the light-emitting units 22 of the light-emitting substrate 2, such as luminance and/or chromaticity (color coordinates). In practice, the holder 13 can be a light-permeable suction holder. The light sensor 15 is disposed above the holder 13, and can include a light analyzer (e.g., a spectrometer).

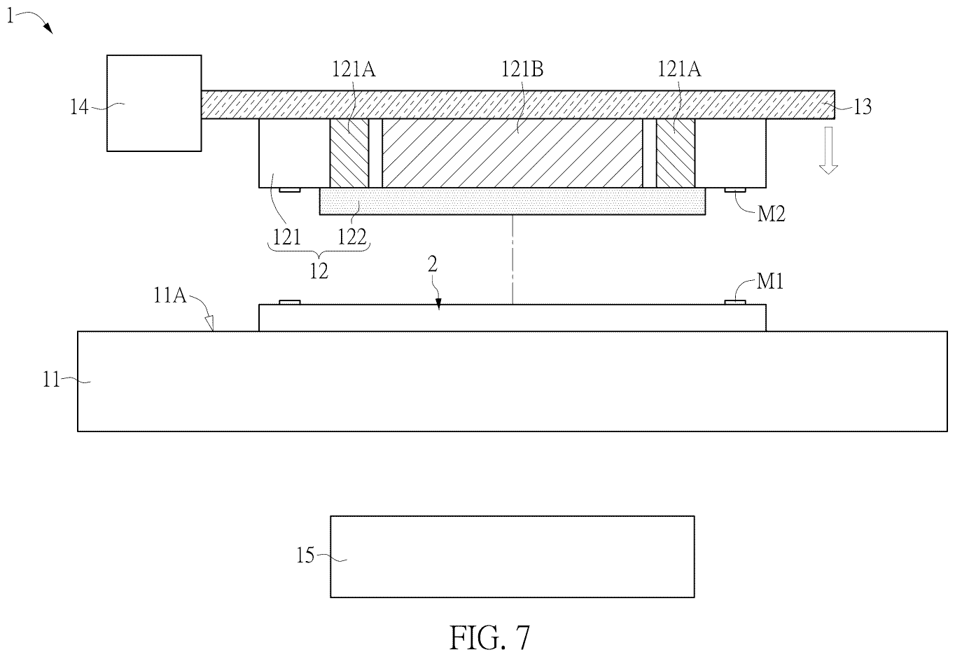

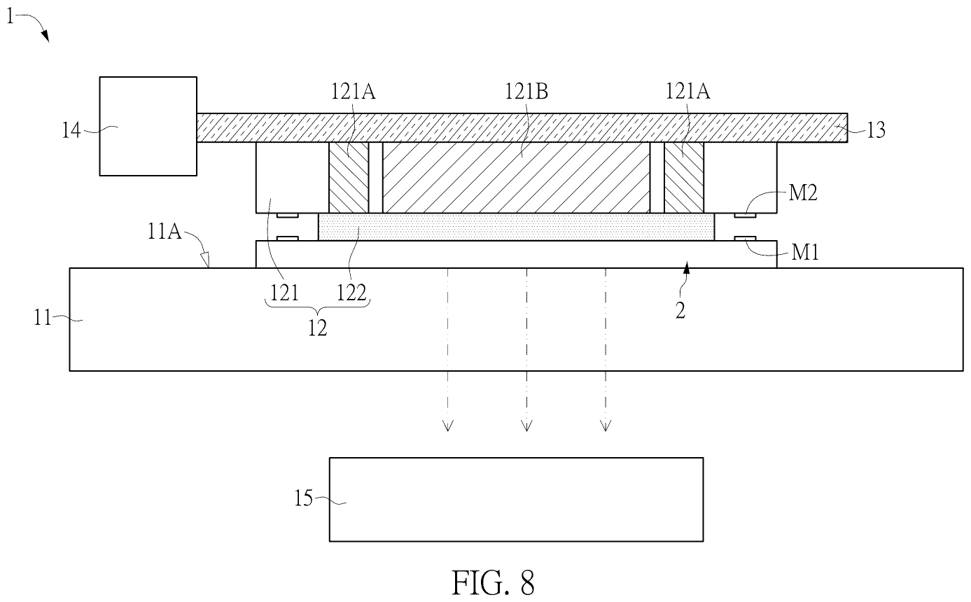

In the test operation of still another possible embodiment of the present disclosure, as shown in FIG. 7 and FIG. 8, the light-emitting substrate 2 to be tested is held on the work stage 11 and remains stationary in the test region 11A. The holder 13 is configured to hold the test assembly 12 above the work stage 11, and to align the test assembly 12 with the light-emitting substrate 2 in the test region 11A. The driver 14 is drivingly connected to the holder 13. Accordingly, the holder 13 can be driven to move the test board 121 toward the light-emitting substrate 2 (i.e., downward), such that the anisotropic conductive layer 122 is pressed between the light-emitting substrate 2 and the test board 121 to establish signal conduction. Furthermore, the light sensor 15 is configured to obtain at least one light-emitting characteristic parameter of each of the light-emitting units 22. For example, the light sensor 15 can be disposed beneath the work stage 11.

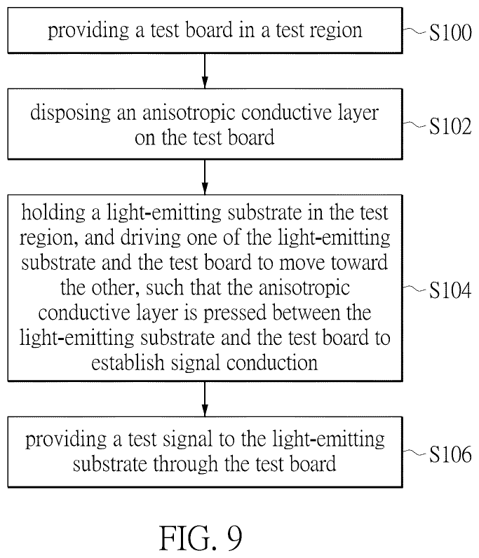

Referring to FIG. 9, the present disclosure further provides a method for testing a light-emitting substrate, which can be implemented by the testing machine described in the foregoing paragraphs. The method of the present disclosure includes: step S100, providing a test board in a test region; step S102, disposing an anisotropic conductive layer on the test board; step S104, holding a light-emitting substrate in the test region, and driving one of the light-emitting substrate and the test board to move toward the other, such that the anisotropic conductive layer is pressed between the light-emitting substrate and the test board to establish signal conduction; and step S106, providing a test signal to the light-emitting substrate through the test board.

Reference is made to FIG. 1 to FIG. 4. In step S104, the test board 121 remains stationary and the light-emitting substrate 2 is driven to move toward the test board 121, such that the anisotropic conductive layer 122 is pressed between the light-emitting substrate 2 and the test board 121. Alternatively, the light-emitting substrate 2 remains stationary and the test board 121 is driven to move toward the light-emitting substrate 2, such that the anisotropic conductive layer 122 is pressed between the light-emitting substrate 2 and the test board 121. Preferably, when the anisotropic conductive layer 122 is pressed between the light-emitting substrate 2 and the test board 121, precise vertical alignment can be achieved by aligning a first alignment mark M1 on the light-emitting substrate 2 with a second alignment mark M2 on the test board 121. Furthermore, in step S106, a light sensor 15 is configured to detect at least one light-emitting characteristic parameter of each of the light-emitting units 22 of the light-emitting substrate 2.

### Beneficial Effects of the Embodiments

In conclusion, the machine and method for testing a light-emitting substrate can shorten testing time, simplify testing operations, reduce testing difficulty, and improve testing accuracy, by virtue of the test board including a first conductive region and a second conductive region formed in accordance with a specific layout design, the anisotropic conductive layer formed on the test board and covering the first conductive region and the second conductive region, and driving one of the light-emitting substrate and the test board to move toward the other, such that the anisotropic conductive layer is pressed between the light-emitting substrate and the test board to establish signal conduction.

More specifically, in the present disclosure, the anisotropic conductive layer is used in combination with the test board having a specially designed conductive pattern layout, and the combination serves as a test assembly for testing the optoelectronic performance of the light-emitting substrates. The anisotropic conductive layer is formed from an anisotropic conductive material, and therefore exhibits electrical conduction in the Z direction and insulation in the X and Y directions. Furthermore, the anisotropic conductive layer is elastic such that, when compressed, it can be in close contact with the test board and the light-emitting substrate to be tested. Therefore, during testing, the anisotropic conductive layer can maintain stable and reliable contact with the conductive structures and provide good electrical conduction without being affected by differences in structural height.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for testing a light-emitting substrate, comprising:
providing a test board in a test region;
disposing an anisotropic conductive layer on the test board;
holding a light-emitting substrate at a position aligned with the test region, and driving one of the light-emitting substrate and the test board to move toward the other, so as to establish signal conduction between the light-emitting substrate and the test board through the anisotropic conductive layer; and
providing a test signal to the light-emitting substrate through the test board, and determining performance of the light-emitting substrate according to one or more of light-emitting characteristics or electrical signals generated by the light-emitting substrate in response to the test signal.

2. The method according to claim 1, wherein, in providing the test board, the test board includes a first conductive region and a second conductive region having different electrical characteristics; wherein, in disposing the anisotropic conductive layer, the anisotropic conductive layer covers the first conductive region and the second conductive region; wherein, in providing the test signal to the light-emitting substrate, the light-emitting substrate includes a first conductivity-type semiconductor layer and a plurality of light-emitting units and a first conductive structure formed on the first conductivity-type semiconductor layer, the first conductive structure is located on one side of the light-emitting units, and each of the light-emitting units includes a light-emitting layer disposed on the first conductivity-type semiconductor layer, a second conductivity-type semiconductor layer disposed on the light-emitting layer, and a second conductive structure disposed on the second conductivity-type semiconductor layer; and wherein the test signal includes a first test signal and a second test signal, the first test signal sequentially passes through the first conductive region of the test board and the anisotropic conductive layer to be transmitted to the first conductive structure of the light-emitting substrate, and the second test signal sequentially passes through the second conductive region of the test board and the anisotropic conductive layer to be transmitted to the second conductive structure of each of the light-emitting units of the light-emitting substrate.

3. The method according to claim 1, wherein providing the test signal to the light-emitting substrate includes obtaining at least one light-emitting characteristic parameter of each of the light-emitting units of the light-emitting substrate through a light sensor.

4. The method according to claim 1, wherein, in the step of establishing the signal conduction between the light-emitting substrate and the test board through the anisotropic conductive layer, the test board remains stationary and the light-emitting substrate is driven to move toward the test board, such that the anisotropic conductive layer is pressed between the light-emitting substrate and the test board.

5. The method according to claim 4, wherein, when the anisotropic conductive layer is pressed between the light-emitting substrate and the test board, a first alignment mark on the light-emitting substrate is aligned with a second alignment mark on the test board.

6. The method according to claim 1, wherein, in the step of establishing the signal conduction between the light-emitting substrate and the test board through the anisotropic conductive layer, the light-emitting substrate remains stationary and the test board is driven to move toward the light-emitting substrate, such that the anisotropic conductive layer is pressed between the light-emitting substrate and the test board.

7. The method according to claim 6, wherein, when the anisotropic conductive layer is pressed between the light-emitting substrate and the test board, a first alignment mark on the light-emitting substrate is aligned with a second alignment mark on the test board.

8. A machine for testing a light-emitting substrate, comprising:
a test region;
a test assembly disposed in the test region, the test assembly including a test board and an anisotropic conductive layer disposed on the test board;
a holder configured to hold a light-emitting substrate in the test region; and
a driver configured to drive one of the light-emitting substrate and the test board to move toward the other, so as to establish signal conduction between the light-emitting substrate and the test board through the anisotropic conductive layer;
the machine is configured to provide a test signal to the light-emitting substrate through the test board, and to determine performance of the light-emitting substrate according to one or more of light-emitting characteristics or electrical signals generated by the light-emitting substrate in response to the test signal.

9. The machine according to claim 8, wherein the test board includes a first conductive region and a second conductive region having different electrical characteristics, and the anisotropic conductive layer covers the first conductive region and the second conductive region; wherein the light-emitting substrate includes a first conductivity-type semiconductor layer and a plurality of light-emitting units and a first conductive structure formed on the first conductivity-type semiconductor layer, the first conductive structure is located on one side of the light-emitting units, and each of the light-emitting units includes a light-emitting layer disposed on the first conductivity-type semiconductor layer, a second conductivity-type semiconductor layer disposed on the light-emitting layer, and a second conductive structure disposed on the second conductivity-type semiconductor layer; and wherein the test signal includes a first test signal and a second test signal, the first test signal sequentially passes through the first conductive region of the test board and the anisotropic conductive layer to be transmitted to the first conductive structure of the light-emitting substrate, and the second test signal sequentially passes through the second conductive region of the test board and the anisotropic conductive layer to be transmitted to the second conductive structure of each of the light-emitting units of the light-emitting substrate.

10. The machine according to claim 8, further comprising a light sensor that is configured to obtain at least one light-emitting characteristic parameter of each of the light-emitting units of the light-emitting substrate.

11. The machine according to claim 8, wherein the holder is a light-permeable suction holder, and the light sensor is disposed above the light-permeable suction holder.

12. The machine according to claim 11, wherein the test board remains stationary in the test region, and the driver is drivingly connected to the holder to move the light-emitting substrate toward the test board, such that the anisotropic conductive layer is pressed between the light-emitting substrate and the test board.

13. The machine according to claim 11, wherein the light-emitting substrate remains stationary in the test region, and the driver is drivingly connected to the test assembly to move the test board toward the light-emitting substrate, such that the anisotropic conductive layer is pressed between the light-emitting substrate and the test board.

14. The machine according to claim 10, further comprising a work stage with the test region, wherein the light sensor is disposed beneath the work stage.

15. The machine according to claim 14, wherein the test board remains stationary in the test region, and the driver is drivingly connected to the holder to move the light-emitting substrate toward the test board, such that the anisotropic conductive layer is pressed between the light-emitting substrate and the test board.

16. The machine according to claim 14, wherein the light-emitting substrate remains stationary in the test region, and the driver is drivingly connected to the test assembly to move the test board toward the light-emitting substrate, such that the anisotropic conductive layer is pressed between the light-emitting substrate and the test board.
